(19) United States
(12) Patent Application Publication
(10) Pub. No.: US 2004/0029196 A1
(43) Pub. Date: Feb. 12, 2004

(54) Epithelial cell lines from gene knockout mice and methods of use thereof

(76) Inventors: Nitin Telang, Pelham Manor, NY (US); Meena Katdare, Jersey City, NJ (US)

Correspondence Address: KLAUBER & JACKSON, 411 HACKENSACK AVENUE, HACKENSACK, NJ, 07601

(21) Appl. No.: 10/335,223

(22) Filed: Dec. 31, 2002

Related U.S. Application Data
(60) Provisional application No. 60/345,730, filed on Dec. 31, 2001.

(51) Int. Cl. G01N 33/574; C12N 5/06

(52) U.S. Cl. 435/7.23; 435/354; 800/3

(57) ABSTRACT

The invention is directed to the development of model systems in which to study epithelial cell transformation and cancer chemoprevention. Accordingly, the present invention provides subculturable epithelial cell lines from gene knockout animals that may be used to advantage in such studies. Also provided are methods for screening chemopreventive agents using the subculturable epithelial cell lines of the invention.

This application claims priority under 35 U.S.C. §119(e) to U.S. Provisional Application No. 60/345,130, filed Dec. 31, 2001.

Pursuant to 35 U.S.C. §202(c) it is acknowledged that the U.S. Government has certain rights in the invention described herein, which was made in part with funds from the National Cancer Institute, Bethesda, Md., grants MAO/RFP#N01-CN-85141, MAO/RFP #N01-CN-05107 and MAO/RFP # N01-CN-25 111.

### FIELD OF THE INVENTION

This invention relates to the field of carcinogenesis and its prevention. More specifically, the invention relates to the development of epithelial cell lines that provide model systems for examining epithelial cell transformation, preventive efficacy of synthetic and naturally occurring compounds, and methods of use thereof.

### BACKGROUND OF THE INVENTION

Several publications and patent documents are referenced in this application in parentheses, for example, in order to more fully describe the state of the art to which this invention pertains. The disclosure of each of these publications and patent documents is incorporated by reference herein.

Gastrointestinal tract cancer and breast cancer are leading causes of morbidity and mortality in the US population (Landis et al., 1998, CA Cancer J. Clin. 48:6-29). It is therefore desirable to develop systems that can be used to identify chemical agents that are likely to have preventive activity against such cancers. Candidate systems include both animal systems and cell culture systems.

### SUMMARY OF THE INVENTION

A need, therefore, exists to develop cell lines and cell culture systems wherein cell lines predestined for cancer can be stably cultured and used as model systems in which to screen and identify chemopreventive agents. Such agents may be used to advantage in therapeutic regimens to modulate the progression of a transformed phenotype. The present invention addresses this need by providing methods with which to establish epithelial cell lines from the histologically normal colon of mice that are predisposed to gastrointestinal carcinogenesis. Also provided are epithelial cell lines generated using these methods and methods of use thereof. These cell lines provide cell-based assay systems suitable for high-throughput screening for chemopreventive agents.

In a first aspect of the invention, a method is provided for screening at least one test agent for chemopreventive efficacy, which comprises the steps of:

(1) providing a subculturable epithelial cell line, which exhibits aberrant cellular proliferation;

(2) exposing cells of the subculturable epithelial cell line to the at least one test agent; and

(3) determining an effect of the at least one test agent on cells of the subculturable epithelial cell lines; wherein the subculturable epithelial cell line is derived from histologically normal non-cancerous epithelial tissue of a gene knock out mouse which has an inactivated gene, the presence of which renders the gene knock out mouse susceptible to development of an epithelial cancer.

In an aspect of the method of the invention, the inactivated gene is a tumor suppressor gene.

In a preferred aspect of the method of the invention, the tumor suppressor gene is adenomatous polyposis coli.

In one embodiment of the method of the invention, the effect of the at least one test agent comprises modulating the number of cells of the subculturable epithelial cell line exhibiting cellular responses indicative of a precancerous or cancerous state.

In a particular embodiment of the method of the invention, such modulation is a decrease in the number of cells exhibiting a precancerous or cancerous state.

In an aspect of the method of the invention, cellular responses indicative of a precancerous or cancerous state are selected from the group consisting of aneuploidy, telomerase re-expression, loss of contact inhibition and anchorage-independent growth.

In an aspect of the method of the invention, a subculturable epithelial cell line is derived from histologically normal noncancerous cells comprising at least one mutation in a tumor suppressor gene.

In an aspect of the method of the invention, a subculturable epithelial cell line is derived from histologically normal noncancerous cells comprising at least one mutation in a tumor suppressor gene and the subculturable epithelial cell line displays at least one precancerous or cancerous marker selected from the group consisting of aneuploidy, telomerase re-expression, loss of contact inhibition and anchorage-independent growth.

In an embodiment of the method of the invention, a subculturable epithelial cell line is preneoplastic.

In one aspect of the method of the invention, a subculturable epithelial cell line comprising at least one mutation in a tumor suppressor gene is an epithelial population of not more than 15 passages.

In an embodiment of the invention, a subculturable epithelial cell line is an epithelial population of at least 15 passages.

In another embodiment of the invention, a subculturable epithelial cell line has one or more mutations in a tumor suppressor gene.

Also provided and included herein are subculturable epithelial cell lines derived from histologically normal non-cancerous epithelial tissue of a gene knock out mouse which has an inactivated gene, wherein the presence of the inactivated gene renders the gene knock out mouse susceptible to development of an epithelial cancer and wherein the subculturable epithelial cell line is an epithelial population at least 5 passages.

In a further aspect of the invention, a subculturable epithelial cell line comprises an inactivated gene which is a tumor suppressor gene.

In a particular aspect of the invention, a subculturable epithelial cell line comprises an inactivated gene which is the adenomatous polyposis coli tumor suppressor gene.

In another aspect of the invention, the subculturable epithelial cell line comprising at least one mutation in a tumor suppressor gene is an epithelial population of at least 15 passages.

In an aspect of the invention, a subculturable epithelial cell line is derived from a knock out mouse having a genotype selected from the group consisting of Apc1638N[+/−] and wild type Apc[+/+]C57COL.

In another aspect of the invention, a subculturable epithelial cell line is an early passage cell line.

In an embodiment of the invention, a subculturable epithelial cell line is of gastrointestinal origin.

In a further embodiment of the invention, a subculturable epithelial cell line is derived from colon.

Also provided are clonal derivatives of the subculturable epithelial cell lines of the invention.

In a particular aspect, a subculturable epithelial cell line is Strang No. 1 Apc [+-] 1638NCOL and derivatives thereof.

In another aspect, a subculturable epithelial cell line is Strang No. 2 1638N-Cl and derivatives thereof.

In yet another aspect, a subculturable epithelial cell line is Strang 1638N Pr₁ cells and derivatives thereof.

Also provided is the subculturable epithelial cell line Strang No. 4 Apc [+/+] C57COL cells, which is derived from normal colonic mucosal epithelium of a mouse having an Apc[+/+] C57COL genotype.

Accordingly, the present invention provides subculturable epithelial cell lines derived from normal colonic mucosal epithelium of mice. Such subculturable epithelial cell lines provide model systems in which to screen agents/compounds to evaluate their potential for use as chemopreventive agents. Chemopreventive agents identified using the methods of the present invention may be used to advantage in the treatment of patients in need thereof.

### DETAILED DESCRIPTION OF THE INVENTION

Gene knockout mice have been engineered that carry genetic mutations or deletions in specific genes, which renders them particularly susceptible to the development of tissue specific cancers. Such mice exhibit accelerated development of organ site specific cancers and are, therefore, useful model systems for studying transformation processes in particular organs. Gene knock out mice have been generated that provide model systems for examining the development of epithelial cancers derived from different organs (Fodde et al. 1994, PNAS USA, 91:8969-8973; Oshima et al, 1995, PNAS USA 92:4482-4486; Su et al, 1992, Science 256:668-670; Moser et al, 1990, Science 247:322-324). In such animals, epithelial cells derived from such organs exhibit enhanced susceptibility to the multistep carcinogenic process.

Of particular interest, gene knockout mice harboring targeted mutations in specific codons of the tumor suppressor gene Apc are predisposed to the development of cancers of epithelial origin. These mutant forms of the Apc gene encode truncated forms of the full length APC protein which comprises of 2,843 amino acids. Such truncated forms of APC proteins include those comprising 474, 716, 850, and 1638 amino acids. The mutation in the tumor suppressor Apc gene results in its loss of function. The presence of such APC mutants in mice predisposes such animals to the development of cancers of the breast and intestine (Fodde et al, 1994, PNAS USA 91: 8969-8973; Oshima et al, 1995, PNAS USA 92:4482-4486; Su et al, 1992, Science 256:668-670; Moser et al., 1990, Science 247:322-324; Bertagnoli et al., 1999, Ann NY Acad. Sci. 847:32-44; Zurcher et al. The Mouse in Biomedical Research: Experimental Biology and Oncology 4:11-35, 1982, Academic Press, New York, N.Y.; Edelmann et al, 1999, Cancer Research 59:1301-1307; Sasai et al., 2000, Carcinogenesis 21:953-950). This carcinogenic process can be modified by dietary manipulation (high fat, low calcium, low Vitamin D, low folic acid), or by administration of pharmacological agents such as nonsteroidal anti-inflammatory drugs (NSAIDS), anti-diabetics, natural polyphenols and phytoaleixins (Bertagnoli et al, 1999, Ann NY Acad. Sci. 847:32-44; Boolbol et al. 1996, Cancer Research 56:2556-2560; Beazer-Barkley et al, 1996, Carcinogenesis 17:1757-1760; Jacoby et al, 2000, Cancer Research 60:1864-1870; Saez et al, 1998, Nature Medicine 4:1058-1061; Lefebvre et al., 1998, Nature Medicine 4:1053-1057).

Inactivating mutations in the human Apc gene or in DNA mismatch repair genes have been associated with a predisposition to clinical familial adenomatous polyposis (FAP) syndrome or to hereditary nonpolyposis colon cancer (HNPCC) syndrome (Zurcher et al., The Mouse in Biomedical Research: Experimental Biology and Oncology 4:11-35, 1982, Academic Press, New York, N.Y.; Jacks et al., 1996, Ann Rev. Genetics 30:603; Groden et al., 1991, Cell 66:589-600; Nishiho et al., 1991, Science 253:665-669; Lothe et al., 1993, Cancer Research 53:5849-5852; Lynch et al., 1993, Gastroenterology 104:1535-1549). Gene knockout mice harboring allelic deletions such as Apc⁴⁷⁴ [+/−], Apc⁷¹⁶ [+/−], Apc 1638N [+/−], Mlh₁ [+/−], Mlh₁ [−/−] and Mlh₁1638 exhibit a high incidence of small intestinal adenoma and adenocarcinoma. (Fodde et al, 1994, PNAS USA 91: 8969-8973; Oshima et al, 1995, PNAS USA 92:4482-4486; Su et al, 1992, Science 256:668-670; Moser et al., 1990, Science 247:322-324; Bertagnoli et al., 1999, Ann NY Acad. Sci. 847:32-44; Zurcher et al., The Mouse in Biomedical Research: Experimental Biology and Oncology 4:11-35, 1982, Academic Press, New York, N.Y.; Edelmann et al, 1999, Cancer Research 59:1301-1307; Sasai et al., 2000, Carcinogenesis 21:953-950). Recent evidence has demonstrated that mice harboring a mutation in the TGF-β receptor gene exhibit accelerated development of colonic adenoma and adenocarcinoma (Zhu et al., 1998, Cell 94: 703-714). Apc^{Min}/+mice administered the PPAR-γ agonist troglitazone or the colon carcinogen Azoxymethane (AOM) also develop colon adenoma. (Saez et al., 1998, Nature Medicine 4:1058-1061; Lefebvre et al., 1998, Nature Medicine 4:1053-1057; Paulsen et al., 2001, Cancer research 61:5010-5015), and Apc^{Min}/Msh₂ double gene knockout mice exhibit a higher incidence of aberrant colonic crypt foci and colon adenomas (Song et al., 2000, Cancer Research 60:3191-3199). Thus, existing evidence from the gene knockout mouse models indicates that the carcinogenic process in the colon may be experimentally induced.

Cultured epithelial cell lines have provided model systems of utility for screening and identifying efficacious chemopreventive agents. Earlier studies were focused on development of reliable in vitro (explant and epithelial cell culture) models to examine the multistep process of breast carcinogenesis. (Telang et al. 1979, PNAS USA 76: 5886-5890; Telang et al. 1982, J. Natl. Cancer Institute, 68:1015-1022; Telang et al., 1990, Cell Regulation 1: 863-872). This approach has identified several molecular, biochemical and cellular mechanisms that are critical for effective cancer prevention (Telang et al. 1992, J. Cell Biochem. 16G: 161-169; Telang et al. 1996, Ann NY Acad. Sci. 784: 277-287; Telang et al. 1997, Environment Health Perspective 105 (suppl.3):559-564; Telang et al. 1997, Proc. Soc. Exp. Bio. Med. 216: 246-252; Telang et al. 1998, British J. Cancer 77: 1549-1554; Katdare et al., 1999, Ann NY Acad. Sci. 889:247-252; Jinno et al, 1999, Carcinogenesis 20: 229-236). Specifically, these experiments identified mechanistic endpoint biomarker assays that quantify the status of pre-neoplastic transformation. Subsequent studies using the developed epithelial cell culture models examined the down-regulation of perturbed biomarkers as a result of exposure to mechanistically distinct classes of chemopreventive agents, thus validating the biomarkers as mechanistic endpoints with which to evaluate the preventive efficacy of several distinct classes of naturally occurring or synthetic compounds (Telang et al. 1996, Ann NY Acad. Sci. 784: 277-287; Telang et al. 1997, Environment Health Perspective 105 (suppl.3):559-564; Telang et al. 1997, Proc. Soc. Exp. Bio. Med. 216: 246-252; Telang et al. 1998, British J. Cancer 77: 1549-1554; Katdare et al., 1999, Ann NY Acad. Sci. 889:247-252; Jinno et al, 1999, Carcinogenesis 20: 229-236).

Nevertheless, the use of epithelial cell line-derived assays for chemopreventive agents has not been without its own limitations. Existing evidence in the literature indicates that epithelial cells from histologically normal small intestinal or colonic mucosa (target tissue for gastrointestinal carcinogenesis) have a limited in vitro life span and are not well suited for subculturing. Long term in vitro survival of an epithelial phenotype has, however, been achieved following stable transfection with oncogenes. Such procedures involve complicated protocols of cell dissociation, use of conditioned media and specialized extracellular matrix substrates (Kalabis et al., 2000, Proc. Amer. Assoc. Cancer Res. 41: 173 (Abstract # 1108); Ohnishi et al., 1999, Biotechniques 27: 978-985; Booth et al., 1995, Epith. Cell Biol. 4:78-86; Whitehead et al, 1987, In Vitro Cell Dev. Biol. 23:436-442; Whitehead et al, 1994, Epith. Cell Biol. 3:119-125; Sevignani et al, 1998, J. Clin. Invest. 101:1572-1580; Grossman et al., 1998, Amer. J. Pathol. 153:53-62; Sevignani et al. 1999, Cancer Res. 59:5882-5886). Epithelial cell lines derived from non-cancerous target tissue or from fully transformed cancer cells are, however, generally not responsive to inhibitory growth regulators or to inducers of differentiation. These cells also lack intact signaling pathways that contribute to normal cellular proliferation and differentiation. Such pathways regulate cell cycling and cell renewal in the gastrointestinal mucosal epithelium in vivo. Moreover, since epithelial cell lines derived from transformed tissue have already progressed beyond the pre-neoplastic and pre-invasive stage of carcinogenesis, such cell lines may be an inappropriate model for identifying chemopreventive agents capable of modulating the early occurring genetic, molecular or biochemical events critical for cellular transformation.

Definitions

As used herein, the terms “histologically normal cells” or “non-cancerous cells” refer to epithelial cells derived from a mouse colon which can be collected from the colon of a healthy mouse, and which can be cultured for an extended period of time without losing their original differentiation characteristics.

As used herein, the terms “neoplastic and “cancerous” are used interchangeably, as are the terms “preneoplastic” and “precancerous”.

As used herein, the term “passage” refers to the process wherein an aliquot of a preconfluent culture of a cell line is used to inoculate a new culture comprised of fresh medium, which is in turn cultured under the appropriate conditions to a desired degree of confluence or saturation. The cell lines are thus traditionally cultured by successive passages in fresh media. The passage number of a cell line may be referred to herein in abbreviated form (e.g., p5 through p25 stands for passages 5 through 25, respectively).

As used herein, the term “subculturable” refers to the ability of a cell line to be passaged repeatedly.

As used herein, “early passage cells” refer to those cell lines subcultured at least 10 times but not more than 20 times.

As used herein, the term “knock out mouse” refers to a mouse in which a gene or genes have been mutated such that the activity of the gene has been reduced or eliminated.

Gene knockout animals, in general, are well known in the art. Moreover, knockout animals in which tumor suppressor genes have been inactivated are commercially available or may be produced by standard methods (see, for example, 20, 21, 23, 31, 38, and 39).

used herein, the term “tumor suppressor gene” refers to a gene or gene product, the activity of which serves to a) maintain normal apoptotic and cell cycle regulatory controls and/or b) inhibit the onset of biochemical intracellular pathways (e.g., cell cycle pathways) that lead to the onset of a transformed phenotype. Some tumor suppressor genes are known to increase the tendency of an animal to develop epithelial cancer, for example, when the gene is inactivated. The tendency of an animal to develop epithelial cancer may be assessed by well known methods. For example, a knockout animal may be compared to a normal animal for this purpose, and both may in addition be exposed to epithelial carcinogens for the comparison (see, for example, 39-42 and references cited in 39-42).

As used herein, “aberrant cellular proliferation” refers to an increase in the number of cells due, in part, to altered cell cycle progression, population doubling time, and/or or decreased apoptosis.

As used herein, “susceptible to development of an epithelial cancer” refers to perturbation of molecular, biochemical or cellular biomarkers that are associated with increased risk for carcinogenic transformation.

As used herein, the terms “cellular responses indicative of a precancerous or cancerous state” or “biomarkers indicative of a precancerous or cancerous state” refer to cellular responses that include, but are not limited to, persistence of aberrant proliferation, altered cell cycle progression, down regulation of apoptosis, aneuploidy, telomerase re-expression, loss of contact inhibition, and/or anchorage-independent growth. Such biomarkers are associated with the carcinogenic transformation. The number of such responses exhibited by a cell and/or the degree to which a cell displays any one of these cellular responses is indicative of the progression of the cell towards a transformed phenotype. A precancerous and/or cancerous epithelial cell, for example, is an epithelial cell which exhibits loss of contact inhibition, aneuploidy, telomerase reexpression and anchorage independent growth.

The degree of loss of contact inhibition, aneuploidy, telomerase re-expression, or anchorage-independent growth, may also be quantified by methods well known in the art. An increase in the degree of loss of contact inhibition may, for example, be evidenced by a persistent increase in cell piling and focus formation. An increase in aneuploidy may be detected by measuring increases in the tetraploid and/or hypertetraploid phenotype of a cell. Telomerase re-expression subsequent to replicative senescence (crisis) may be measured by an increase in the addition of 5′-T-T-T-A-G-G-G-3′ telomeric nucleotide repeat sequences to the chromosomal ends of replicating DNA. An increase in anchorage independent growth may be determined using colony formation assays which detect the number of non-adherent colonies formed.

Exemplary Assays for Detecting/Measuring Cellular Responses Indicative Carcinogenic Transformation

Loss of contact inhibition: Microscopic examination of confluent cultures for the presence of foci of ‘piled up’ cells. The multicellular foci are distinguishable from adjacent single cells forming a monolayer.

Aneuploidy: Fluorescence assisted cell sorting and flow cytometry of cells stained with DNA binding fluorescent dyes. Diploid and aneuploid cells accumulate as distinct peaks on the DNA histograms obtained from flow cytometric analysis.

Telomerase: Telomeric repeat amplification protocol (TRAP) assay is a cell free assay that monitors polymerase-mediated addition of nucleotide repeat sequences to DNA. The addition of nucleotide repeats is dependent on the presence of telomerase enzyme. Gel electrophoretic separation of DNA reveals the presence of DNA ladder which provides a positive indicator of telomerase activity/expression. See also Ohyashiki et al. Trends Genet. 12: 395-396, 1996. Kits are commercially available for such analyses, for example, TRAPEZE telomerase detection kit (Oncor, Gaithersburg, Md.).

Anchorage-independent growth: The ‘soft’ agar growth assay monitors the ability of cells to form non adherant colonies. Single cells suspended in 0.33% agar are overlaid on a basement matrix. These cultures are maintained for about 10-14 days and resulting colonies are counted.

Such methods are described in detail in a number of standard laboratory manuals. See, for example, Culture of Animal Cells: A Manual of Basic Technique. R. Ian Freshney. Wiley-Liss, New York. 1987, the entire contents of which is incorporated herein by reference.

Preferred cells for use in the screening method of the invention are epithelial cells that are histologically normal noncancerous cells which harbor one or more mutations in at least one tumor suppressor gene. Such mutations may result in a loss of function with regard to the activity of the tumor suppressor gene. Examples of tumor suppressor genes include, but are not limited to the Apc gene, p53 gene, pRb gene, BRCA-1 gene and BRCA-2 gene. The present invention is not, however, limited to epithelial cell lines derived from the normal noncancerous tissue of mice comprising inactivating mutations in these tumor suppressor genes. The present invention comprises cell lines derived from knock out mice in which any tumor suppressor gene has been inactivated. Assays for determining that a gene is a tumor suppressor gene are known in the art (Jacks et al. 1996, Ann Rev Genetics 30:603).

A cell may be designated a histologically normal noncancerous cell by confirming the absence of markers indicative of a precancerous and/or cancerous state. Such markers include, but are not limited to, aneuploidy, telomerase re-expression, loss of contact inhibition and anchorage-independent growth.

The screening method described herein encompasses exposure of a subculturable epithelial cell line of the present invention to a potential chemopreventive for an appropriate length of time. The duration of exposure may be determined by a skilled artisan based on the chemopreventive agent, the concentration of the agent, and the passage number of the cell line. See Telang et al, 1997, Proc. Soc. Exp. Biol. Med. 216:246-252. Typically, the duration of exposure is 1 to 3 weeks at 37° C. A duration of 1-3 weeks provides sufficient time for the cell lines of the invention to exhibit cellular responses indicative of a precancerous or cancerous state such as, but not limited to, aneuploidy, telomerase re-expression, loss-of contact inhibition and anchorage-independent growth. Cells that are at passage 10, for example, generally progress to a passage number in the range 11 to 13 during a 1 to 3 week incubation. Cells that are at passage 20 generally progress to a passage number in the range 21 to 23 during a 1 to 3 week-incubation. For cells that are already preneoplastic (e.g., at 20 passages), a typical duration time for incubation with a test agent is about 5 weeks at 37° C., during which time the cells progress to about passage 25.

The present invention is directed to mammalian cell lines (e.g., mouse cell lines) that harbor at least one mutation in a tumor suppressor gene (e.g. Apc1638N [+/−]). Such mutated cell lines may be derived from mammals that are either homozygous or heterozygous for such mutations. In particular, the invention is directed to cell lines comprising inactivated tumor suppressor genes which are of gastrointestinal origin (e.g., the colon). The 1638N mouse strain, which has the Apc1638[+/−] genotype, may be used to advantage as a source for such cell lines. Highly preferred cell lines are early passage cells. Examples of early passage cells are:

Strang No. 1: Apc[+/−] 1638NCOL cells

Strang No. 2: 1638NCl₁ cells

Strang No. 3: 1638N Pr₁ cells

Strang No. 4: Apc [+/+] C57COL cells

The above listed cell lines will be deposited with the ATCC.

The cell line Strang No. 1 is also referred to herein as the 1638NCOL (Apc [+/−]) cell line. The cell line Strang No. 4 is a control cell line. The cell lines Strang Nos. 1 and 4 are epithelial cell lines established from histologically normal colonic mucosal epithelium of Apc[+/−]1638N and Apc[+/+]C57BL/6J mice, respectively. The cell lines Strang Nos. 2 and 3 are clonal derivatives of Apc[+/−]1638N cells that exhibit preneoplastic transformation in vitro and tumorigenic transformation in vivo.

Cell lines or cells that are homologs or derivatives of Strang No. 1-4 cell lines are also encompassed by the present invention. A homolog of a first cell line is a second cell line derived from a different donor, but generated using similar methods as those used to generate the first cell line. Derivatives of a cell line are clonal isolates of the cell line. Any cell of this application may therefore be a homolog or a derivative of the foregoing cells or cell lines.

1638NCOL Apc[+/−] cells were derived from histologically normal noncancerous colonic epithelial tissue of strain 1638N mice with the Apc [+/−] genotype.

Selection of Tissue Culture Medium

To identify the most appropriate medium for long-term survival and repeated frequent subculturing of the epithelial cells, primary cultures were maintained separately in several commercially available basal tissue culture media such as KBM/MEM (vendor: Clonetics-Biowhitaker, San Diego, Calif.; GIBCO-BRL, Grand Island, N.Y.), WME (vendor: GIBCO-BRL) and DME/F12 (vendor: GIBCO-BRL). The basal media were supplemented with fetal bovine serum, selected polypeptide hormones (insulin, dexamethasone, hydrocortisone) and growth factors. Within 7-10 days all cultures except those maintained in supplemented DME/F12 medium exhibited massive apoptosis. Thus, DME/F12 was proven to be most effective in supporting long-term survival of epithelial cell cultures.

Based on previous experiments in which conditions were optimized for explant cultures derived from rodent colon (3), serum, insulin and dexamethasone were tested as additives to DME/F 12 medium. DME/F 12 medium supplemented with 10% fetal bovine serum (GIBCO-BRL) 10 ug/ml insulin, and 1 μM dexamethasone (Sigma Chemical Co., St. Louis) was found to be the most appropriate medium for long term survival, repeated subculturing and selective growth of colonic epithelial cells from gene knockout mice. The ingredients of DME/F 12 medium (Life Technologies, Catalogue No. 1 1330) are in Table 5.

Adaptation of Cells to Culture

The number of passages is calculated as follows: Upon creation of the primary culture from epithelial tissue, the cells are not yet considered to have undergone a passage. Each time the cells are trypsinized and reseeded in a cell culture vessel (e.g., a glass or plastic bottle) the number of passages increases by one. In the present case, trypsinization is delayed until the cells have reached about 70% confluence and the trypsinized cells are reseeded at a cell density of about one-fifth or one-tenth that of the trypsinized culture.

It is preferred that the cells undergo at least 5 passages before being used in an assay for a chemopreventive agent. During those passages, the cells adapt to cell culture. Any of a number of criteria may be used to monitor cell adaptation. A preferred criterion is the rate of cell growth as defined by the cell number at 70% confluency, relative to that of the initial seeding density. Cell number is determined by routine haemocytometer cell count. The rate of cell growth increases with each passage number up to approximately the fifth passage after which it stays roughly constant.

Control cells are similarly allowed to adapt to at least the fifth passage. Subsequent to about 15 passages, control cells (“normal cells”) begin to lose their ability to survive in culture.

Pharmaceutical Compositions

Chemopreventive compounds/agents identified using the methods of the present invention may be incorporated into pharmaceutical compositions that may be delivered to a subject in need thereof. In a particular embodiment of the present invention, pharmaceutical compositions comprising such agents may be administered to a recipient in a therapeutically effective amount to inhibit the onset and/or progression of disease in the subject. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient, either alone or in combination with other agents, angiogenic modulators, drugs (e.g., antibiotics) or hormones. In preferred embodiments, the pharmaceutical compositions also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., 18th Edition, Easton, Pa. [1990]).

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art. The pharmaceutical compositions of the present invention may be manufactured in any manner known in the art (e.g., by means of conventional mixing, dissolving, granulating, dragee-making, levigating, emulsifying, encapsulating, entrapping, or lyophilizing processes).

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they may be placed in an appropriate container and labeled for treatment. For administration of chemopreventive agent/compounds, such labeling would include amount, frequency, and method of administration.

Pharmaceutical compositions suitable for use in the invention include compositions wherein the active ingredients are contained in an effective amount to achieve the intended therapeutic purpose. Determining a therapeutically effective dose is well within the capability of a skilled medical practitioner using the techniques provided in the present invention. In vivo imaging technologies including, but not limited to, x-rays and magnetic resonance imaging (MRI) may be used to visualize reduction in the disease state of a patient, although other techniques known in the art may also be used. A reduction in the disease state of a patient may refer, for example, to a reduction in the tumor burden or the size of a tumor in a treated patient. Accordingly, the information derived from imaging techniques may be used to determine the therapeutic efficacy of a compound(s) so administered. Such information may be used by a skilled practitioner to optimize the therapeutic regimen for the treatment of a patient with a particular disease or an individual patient.

Therapeutic doses will depend on, among other factors, the age and general condition of the subject, the type of disease, and the severity of the disease burden. Thus, a therapeutically effective amount in humans will fall in a relatively broad range that may be determined by a medical practitioner based on the response of an individual patient to treatment with a chemopreventive agent identified using the methods of the present invention.

Administration

Chemopreventive agents identified using the methods of the present invention may be administered to a patient by a variety of means (see below) to achieve and maintain a therapeutically effective level of the agent. One of skill in the art could readily determine specific protocols for using agents so identified for the therapeutic treatment of a particular patient.

Chemopreventive agents identified using the methods of the present invention may be administered to a patient by any means known. Direct delivery of the pharmaceutical compositions in vivo may generally be accomplished via injection using a conventional syringe, although other delivery methods such as convection-enhanced delivery are envisioned (See e.g., U.S. Pat. No. 5,720,720, incorporated herein by reference). In this regard, the compositions may be delivered subcutaneously, epidermally, intradermally, intrathecally, intraorbitally, intramucosally, intraperitoneally, intravenously, intraarterially, orally, intrahepatically or intramuscularly. Other modes of administration include pulmonary administration, suppositories, and transdermal applications. In particularly preferred embodiments, the compositions may administered intravenously in an artery which provides blood flow to an organ for which treatment is desired. A clinician specializing in the treatment of patients with cancer may determine the optimal route for administration of the chemopreventive agents based on a number of criteria, including, but not limited to: the condition of the patient and the type of cancer afflicting the patient.

one aspect, chemopreventive agents identified using the methods of the present invention may be used to treat a patient with a disorder characterized by aberrant cellular proliferation. In a preferred aspect of the present invention, such agents may be used to treat a patient with a cancer. Such cancers include, but are not limited to, cancers of gastrointestinal origin (e.g., small intestine and colon), breast, prostate, bladder etc., depending upon the availability of appropriate cell lines.

From the foregoing discussion, it can be seen that chemopreventive agents identified using the methods of the present invention may be used in the treatment of a patient with a hyperproliferative disorder, such cancer. In a particular embodiment, such chemopreventive agents may be used to treat a patient with colon cancer.

The following examples are provided to illustrate certain embodiments of the invention. They are not intended to limit the invention in any way.

### EXAMPLE I

The present invention provides methods that may be used to advantage to establish epithelial cell lines from the histologically normal colons of gene knockout mice predisposed to gastrointestinal carcinogenesis. Such methods are based on technology optimized for the development of breast epithelial cell lines (Telang et al., 1979, PNAS USA 76:5886-5890; Telang et al., 1990, Cell Regulation 1: 863-872; Telang et al. 1992, J. Cell Biochem. 16G: 161-169; Telang et al. 1996, Ann NY Acad. Sci. 784: 277-287; Telang et al. 1997, Environment Health Perspective 105 (suppl.3):559-564; Telang et al. 1997, Proc. Soc. Exp. Bio. Med. 216: 246-252; Telang et al. 1998, British J. Cancer 77: 1549-1554; Katdare et al., 1999, Ann NY Acad. Sci. 889:247-252; Jinno et al, 1999, Carcinogenesis 20: 229-236).

Technical limitations specific for establishing subculturable epithelial cell lines from gastrointestinal tissue include, but are not limited to i) the existence of defined proliferative/cytodifferentiative compartments in the organ; ii) the presence of microflora; and iii) the complexities related to a defined stem cell population. The presence of strict compartmentalization leads to a mixed population of stem cells and terminally differentiated cells in the primary culture. Most conventional tissue culture media are formulated to support proliferatively active phenotypes, but are not well suited to maintaining a stem cell phenotype or a terminally differentiated phenotype. In the breast, the proliferative/cytodifferentiative gradient is not well defined. Moreover, the presence of bacterial microflora in the gastrointestinal tract but not in the breast, renders the former more susceptible to contamination.

As described herein, the cell culture technology has been successfully optimized and effectively validated to reproducibly propagate target epithelial cells from histologically normal noncancerous colon tissue of mice expressing Apc [+/+] and Apc 1638N [+/−] genotypes. The post-immortalized 1638NCOL cells at p21 were evaluated in an anchorage-independent growth assay, and cells from a single anchorage-independent colony were clonally expanded. This cell line was designated as 1638N-C1₁. These cloned cells were subsequently assessed in an in vivo tumorigenicity assay, and cells derived from a single primary tumor were re-established in culture. This cell line was designated as 1638N-Pr₁. (Table 1).

Representative cell cultures of C57COL (wild type Apc [+/+]) have been propagated in vitro for at least 25 passages. 1638NCOL (mutant Apc1638N [+/−]) cell line has been propagated in vitro for at least 25 passages (sub cultures). Early passages (p5) and late passages (p20) have been evaluated for changes in growth rate, cell cycle progression, cellular apoptosis, ploidy and cytodifferentiation. Characterization of the two cell lines has been completed using quantitative endpoint biomarker assays as indicated (Table 2).

The 1638N COL (Apc [+/−] cells used to obtain the data in Tables 2, 3, and 4 were at passage 5, 10, 16, and 18, respectively. The C57COL Apc[+/+] cells used to obtain the data in Table 2 were at passage 5, 10, 16, and 24.

Differential susceptibility of C57COL (wild type Ape [+/+]), the control cell line, and 1638NCOL (Apc [+/−]) to growth inhibition by mechanistically distinct prototype chemopreventive test compounds was evaluated in a conventional cell survival assay, while the preventive efficacy of the test compounds was evaluated using optimized quantitative flow cytometry-based cell cycle analysis. Optimization of flow cytometry based cell cycle analysis is published (7, 8, 10, 11) and is needed in view of batch-to-batch variations in commercially available fluorescein isothiocyanate-conjugated antibodies.

Selective susceptibility of Ape [+/−] 1638NCOL cells to a prototype chemopreventive agent sulindac (SUL) was examined by comparing its effect on Ape [+/+] C57COL and Ape [+/−] 1638NCOL cells. The cytostatic growth arrest response, evidenced by a decrease in cell population doubling, was about 2-3 fold higher in Ape [+/−] cells as compared to that of Ape [+/+] cells (Table 3).

The dose response and efficacy studies involved treatment of cell cultures with five log μM concentrations of each compound (0.01, 0.1, 1.0, 10.0 and 100.0 μM). Treatment with low doses of some of the chemopreventive agents (less that 1 μM) resulted in induction of cytodifferentiation as evidenced by the appearance of a secretory phenotype (acid mucopolysaccharide and MUCl positivity) and by increased immunoreactivity to RAR-β RXR-β, and PPAR-γ proteins (data not shown). The cytostatic effect of treatment with high doses (100 μM) was associated with down regulated cell population doubling, altered aneuploid G₀/G₁: S+G₂/M ratio, and decreased number of anchorage independent colonies (Table 4).

The data generated from experiments presented in Tables 2-4 demonstrate that epithelial cells derived from mice that harbor mutations in the tumor suppressor Ape gene exhibit hyperproliferation, aberrant cell cycle progression, and anchorage-independent colony formation. These data show that the present quantitative endpoint biomarker assays provide a means for evaluating the status of cell proliferation, cell cycle regulation and anchorage-independent growth. The alterations demonstrated in Tables 2-4 are similar to those previously documented in preneoplastic cells derived from breast (4, 6-11). The data in Tables 2-4 also revealed that the perturbed biomarkers in cells derived from Ape gene knockout mice are responsive to modulation by mechanistically distinct prototype chemopreventive test compounds.

The responsiveness of the epithelial cell lines of the present invention to mechanistically distinct prototype chemopreventive test compounds validates the use of these cell lines as preclinical model systems in which to evaluate new pharmaceuticals or nutraceuticals for their ability to prevent the onset and/or progression of colon carcinogenesis. Such preclinical screening assays may be used to advantage to identify effective compounds that can be further evaluated in vivo using animal models and conventional clinical trials. One of skill in the art can use the results of experiments from the present cell culture models to determine doses for testing in humans from extrapolation of IC₅₀ values on body weight or surface area basis.

The instant invention provides an experimental model system in which to evaluate synergistic and/or additive interactions of combinations of mechanistically distinct compounds. It is understood by those of skill in the art that individual compounds may be more efficacious when administered in combination, in part, due to the interactions of distinct molecular, biochemical, and cellular targets of action (27). Such combinatorial approaches provide valuable information regarding optimal combinations of compounds and appropriate concentrations thereof, preferred order of addition, and timing of administration supported by evidence for enhanced efficacy relative to that by independent agent, in part, due to distinct molecular, biochemical or cellular targets of action (27).

Methods and Materials

Isolation of epithelial cells from colonic tissue of gene knockout mice. Histologically normal non-cancerous target tissue derived from the colon of 4-6 week old gene knockout mice (obtained as described in 20, 36) was excised under sterile conditions. The tissue was separately minced on ice and incubated in a 1:1 v/v mixture of 0.01% collagenase+0.01% Hyaluronidase with shaking at 37° C. for about 10-15 minutes. The tissue digest was washed/resuspended thrice with antibiotic containing basal medium, followed by a centrifugation at 1000 rpm (room temperature). The final pellet was resuspended in growth medium (tissue culture medium supplemented with serum, growth factors, hormones and antibiotics) as previously described (4, 6-11). Initially, the primary cultures were fed with fresh medium once a week. After about 1-4 months, visible epithelial colonies were differeritially trypsinized and selectively propagated. The epithelial cells were subcultured for at least five times and purified epithelial cells were stored in liquid nitrogen bank. This protocol was used to establish the cell lines from the Apc [+/+] and Apc [+/−] mice listed in Table 1.

Characterization of Epithelial Cell Lines for Growth kinetics, Cell Cycle Progression, and Anchorage Independent Growth.

The cell lines were characterized for expression of epithelium specific proteins (cytokeratins), Apc gene product (full length, as opposed to truncated forms which are differentially reactive to carboxyl and amino terminal Apc antibodies), and cytodifferentiation (MUC1, acid mucopolysaccharide, alkaline phosphatase) using standard assays for immunocytochemical analysis. The growth kinetics were determined based on the time-dependent increase in the number of viable cells relative to the initial seeding density. The cell cycle progression and anchorage-independent growth were determined using fluorescence assisted flow cytometry and colony forming assays, respectively, as previously described (4, 7-11). The expression profile of specific gene products (proteins) involved in the regulation of cell cycle progression, apoptosis, and differentiation was also quantified using recently optimized cellular epifluorescence assays (10, 11).

Preventive Efficacy of Natural and Synthetic Test Compounds.

The endpoint biomarkers indicative of proliferation, differentiation, apoptosis, cell cycle progression and anchorage-independent growth were altered in the cell lines derived from the gene knockout mice as compared to those derived from wild type Apc [+/+] mice. Mechanistically distinct classes of synthetic chemopreventive agents, administered at the maximum cytostatic concentrations, down regulated these endpoint biomarkers in the cell lines derived from Apc [+/−] mice. These biomarkers represent surrogate endpoints for carcinogenic transformation (5-11). Alterations in biomarker expression in such model systems may, therefore, provide an accurate determination of the chemopreventive efficacy of new compounds/agents. Compounds/agents suitable for screening in such model systems include, but are not limited to, synthetic pharmaceuticals and phytochemicals or nutraceuticals extracted from natural sources.

TABLE 1
Colon Epithelial Cell Culture Model from Gene Knockout Mouse

| | | | Maintenance in |
|---|---|---|---|
| Cell line | Genotype | Origin | Culture |
| C57COL | Apc [+/+] | Colon | p5-p25 |
| 1638NCOL | Apc [+/−] | Colon | p5-p25 |
| 1638Cl₁ | | Anchorage- independent colony | p5 |
| 1638-Pr₁ | | Tumor | p3 |

TABLE 2
Biomarker Expression in Colon Epithelial Cell Lines

| | Cell Line | | | |
|---|---|---|---|---|
| | | | | 1638- |
| Biomarker | C57COL | 1638NCOL | 1638-Cl1 | Cl1/Pr1 |
| Contact inhibition of growth | + | − | − | − |
| Replicative Senescence | p10-p16 | p10-p16 | − | − |
| Telomerase Reexpression | p24 | p18 | NA | NA |
| Population Doubling Time | 34 hr | 17 hr | ND | ND |
| Aneuploidy | 0% | 20% | 70% | 90% |
| Anchorage-independent | − | +p20 | + | + |
| growth | | | | |
| Tumorigenicity | − | +p20 | + | + |

ND: Not done, NA: Not applicable

TABLE 3
Cytostatic Growth Arrest of Apc [+/−] 1638NCOL Cells by Sulindac
(SUL)

| | | Cell Population Doubling at Day 5 Post-Seeding | | | |
|---|---|---|---|---|---|
| | | C57COL | % | | % |
| Treatment | | (Apc [+/+]) | Inhibition | 1638NCOL (Apc [+/−]) | Inhibition |
| EtOH | 0.1% | 3.9 ± 0.7 | — | 7.3 ± 0.3 | — |
| SUL | μM | | | | |
| | 1 | 3.5 ± 0.4 | 10.2 | 4.8 ± 0.6 | 34.2 |
| | 10 | 3.3 ± 0.7 | 15.4 | 4.4 ± 0.4 | 39.7 |
| | 100 | 1.1 ± 0.5 | 71.8 | 2.3 ± 0.3 | 68.5 |

TABLE 4
Efficacy of Chemopreventive Agents on Apc [+/−] 1638NCOL Cells

| | | Biomarker | | |
|---|---|---|---|---|
| | | | | Anchorage- |
| | | Cell Population | Aneuploid | independent |
| Agent | Concentration | Doubling | G₀/G₁:S + G₂/M ratio | colonies |
| EtOH (solvent) | 0.1% | 9.9 ± 0.3 | 7.7 ± 0.6 | 17.8 ± 2.5 |
| 9cisRA | 100 μM | 4.6 ± 0.1 | 18.5 ± 0.2 | 4.4 ± 1.2 |
| DFMO | 100 μM | 3.1 ± 0.1 | 1.5 ± 0.6 | 0.3 ± 0.2 |
| SUL | 100 μM | 3.8 ± 0.2 | 13.9 ± 0.5 | 0.5 ± 0.3 |
| OLT | 100 μM | 8.4 ± 0.4 | 7.2 ± 0.1 | 15.8 ± 1.8 |

TABLE 5
DMEM/F12 medium

| Component | Concentration (mg/L) |
|---|---|
| CaCl₂(anhyd) | 116.60 |
| CuSO₄.5H₂0 | 0.0013 |
| Fe(NO₃)₃.9H₂0 | 0.05 |
| FeSO₄.7H₂0 | 0.417 |
| KCl | 311.80 |
| MgCl₂ | 28.64 |
| MgSO₄ | 48.84 |
| NaCl | 6995.50 |
| NaHCO₃ | 1200.00 |
| NaH₂PO₄.H₂0 | 62.50 |
| Na₂HPO₄ | 71.02 |
| ZnSO₄.7H₂0 | 0.432 |
| D-Glucose | 3151.00 |
| HEPES | 3574.50 |
| Na Hypoxanthine | 2.39 |
| Linoleic Acid | 0.042 |
| Lipoic Acid | 0.105 |
| Phenol Red | 8.10 |
| Sodium Putrescine.2HCl | 0.081 |
| Sodium Pyruvate | 55.00 |
| L-Alanine | 4.45 |
| L-Arginine.HCl | 147.50 |
| L-Asparagine.H20 | 7.50 |
| L-Aspartic Acid | 6.65 |
| L-Cysteine.H₂0 | 17.56 |
| L-Cysteine.2HCl | 31.29 |
| L-Glutamic Acid | 7.35 |
| L-Glutamine | 365.00 |
| Glycine | 18.75 |
| L-Histidine HCl.H₂0 | 31.48 |
| L-Isoleucine | 54.47 |
| L-Leucine | 59.05 |
| L-Lysine HCl | 91.25 |
| L-Methionine | 17.24 |
| L-Phenylalanine | 35.48 |
| L-Proline | 17.25 |
| L-Serine | 26.25 |
| L-Threonine | 53.45 |
| L-Tryptophan | 9.02 |
| L-Tyrosine.2Na.H₂0 | 55.79 |
| L-Valine | 52.86 |
| Biotin | 0.0035 |
| D-Ca pantothenate | 2.24 |
| Choline Chloride | 8.98 |
| Folic Acid | 2.65 |
| I-Inositol | 12.60 |
| Niacinamide | 2.02 |
| Pyridoxine H1 | 2.031 |
| Riboflavin | 0.219 |
| Thiamine H1 | 2.17 |
| Thymidine | 0.365 |
| Vitamin B₁₂ | 0.68 |

### EXAMPLE II

The results shown in the Tables 6-9 were generated essentially as described in Example I. These data confirm and extend the findings of Example I and examine further the effect of additional agents on the growth properties and phenotypic characteristics of the cell lines examined. As shown hereinbelow, the subculturable epithelial cell lines of the present invention provide model systems for the screening of chemopreventive agents. The utility of such lines is underscored by the results observed in experiments using known chemopreventive agents, such as a retinoid receptor modulator, 9cis retinoic acid (9cis RA); a omithine decarboxylase inhibitor, difluoromethylornithine (DFMO); an antiestrogen, Tamoxifen (TAM); an alkaline-phosphatase dependent radio protector, Amifostine (AMF); a phase II enzyme inducer, Oltipraz (OLT); and a non-steroidal anti-inflammatory drug, Sulindac (SUL). These chemopreventive agents are commercially available from Sigma Chemical Co. (St. Louis, Mo.).

Exemplary cell lines of the present invention also include the Mlh1COL and Mlh1/1638N COL cell lines. These lines were developed from mice that exhibit defective expression of the Mlh1 gene, which is involved in DNA repair. Such cell lines may be used to advantage in high throughput screening assays to identify chemopreventive agents as described herein.

TABLE 6
Growth characteristics of Apc [+/+] and Apc [+/−] mouse colonic
epithelial cells.

| Cell line | | | Saturation density | Population | Cell cycle |
|---|---|---|---|---|---|
| progression | Genotype | Aneuploidy (%) | (×10⁵)^{a} | doublings^{a} | (G₀/G₁: S + G₂/M)^{a} |
| C57/COL | Apc [+/+] | 0 | 9.6 ± 0.5^{b} | 5.2 ± 0.5^{d} | 3.9 ± 0.2^{f} |
| 1638N/COL | Apc [+/−] | 20 | 36.6 ± 1.4^{c} | 10.4 ± 0.5^{e} | 2.2 ± 6^{g} |

^{a}Mean ± SD, n = 3;
^{b-c, d-e}p = 0.004;
^{f-g}p = 0.03.

TABLE 7
Response of Apc [+/−] 1638NCOL cells to chemo-preventive agents.

| | | Response | | |
|---|---|---|---|---|
| | Concentration | | Growth | |
| Agent | (μM) | No effect | arrest (%) | Toxicity^{b} |
| 9cisRA | 0.01 | + | | |
| | 0.1-10 | | 30 | |
| | 100 | | 60 | |
| DFMO | 0.01-1.0 | | 30 | |
| | 10 | | 50 | |
| | 100 | | 70 | |
| TAM | 0.01 | + | | |
| | 0.1-10 | | 20-30 | |
| | 100 | | | + |
| AMF | 0.01 | + | | |
| | 0.1-10 | | 25-40 | |
| | 100 | | | + |
| OLT | 0.01 | + | | |
| | 0.1-10 | | 2 | |
| | 100 | | 25 | |
| SUL | 0.01 | + | | |
| | 0.1-10 | | 30 | |
| | 100 | | 70 | |

^{a}No effect <20% inhibition of surviving cell population;
^{b}Toxicity, surviving cell population lower than initial seeding density.

TABLE 8
Cytostatic growth arrest of Apc [+/−] 1638N/COL cells by
chemopreventive agents.

| | | | No. of | |
|---|---|---|---|---|
| | | Growth | population | |
| Agent | Concentration | arrest (%) | doublings | % Change^{a} |
| Et0H | 0.1% | — | 9.9 ± 3^{b} | — |
| | μM | | | |
| 9cisRA | 1 | 30 | 6.7 ± 0.2 | −32.3 |
| | 100 | 60 | 4.6 ± 0.1^{c} | −53.5 |
| DFMO | 1 | 30 | 6.9 ± 0.1 | −30.3 |
| | 100 | 70 | 3.1 ± 0.1^{c} | −68.7 |
| TAM | 1 | 20 | 7.6 ± 0.4 | −23.2 |
| | 10 | 30 | 7.1 ± 0.2 | −28.3 |
| AMF | 1 | 25 | 8.2 ± 0.2 | −17.2 |
| | 10 | 40 | 6.2 ± 0.3 | −37.4 |
| OLT | 1 | 2 | 10.7 ± 0.2 | +8.1 |
| | 100 | 25 | 8.4 ± 0.4 | −15.1 |
| SUL | 1 | 30 | 7.6 ± 0.3 | −23.2 |
| | 100 | 70 | 3.8 ± 0.2^{c} | −61.6 |

^{a}Exp.con/con ×100;
^{b}Mean ± SD, n = 3 per treatment group;
^{b-c}p = 0.01.

TABLE 9
Effects of chemopreventive agents on aneuploid cell cycle progression
of Apc [+/−] 1638N/COL cells.

| Agent | Concentration | G₀/G₁:S + G₂/M ratio^{a} | Cell cycle phase |
|---|---|---|---|
| Et0H | 0.1% | 7.7 ± 0.6^{b} | |
| 9cisRA | μM | | |
| | 1 | 13.8 ± 0.5^{c} | G₁ arrest |
| | 100 | 22.1 ± 0.6^{c} | G₁ arrest |
| DFMO | 1 | 15.8 ± 0.6^{c} | G₁ arrest |
| | 100 | 1.5 ± 0.6^{d} | S,G₂M arrest |
| TAM | 1 | 2.7 ± 0.9^{d} | S,G₂M arrest |
| | 10 | 1.8 ± 0.8^{d} | S,G₂M arrest |
| AMF | 1 | 9.6 ± 0.7 | |
| | 10 | 10.9 ± 0.6^{c} | G₁ arrest |
| OLT | 1 | 1.0 ± 0.2^{d} | S,G₂M arrest |
| | 100 | 1.2 ± 0.1^{d} | S,G₂M arrest |
| SUL | 1 | 6.6 ± 0.4^{d} | |
| | 100 | 13.9 ± 0.8^{c} | G₁arrest |

^{a}Means ± SD, n = 3 per treatment group;
^{b-c}p = 0.001;
^{b-d}p = 0.001.

### REFERENCES

The following disclosures are incorporated herein by reference:

1. Landis S H et al. CA Cancer J. clin. 48: 6-29, 1998.

2. Telang N T, et al. Proc. Natl. Acad. Sci. USA 76: 5886-5890, 1979

3. Telang N T, Williams GM J. Natl. Cancer Inst. 68: 1015-1022, 1982

4. Telang N T et al. Cell Regulation 1: 863-872, 1990

5. Telang N T et al. J. Cell Biochem. 16G: 161-169, 1992.

6. Telang N T Ann NY Acad. Sci. 784: 277-287, 1996

7. Telang N T et al. Environment Health Perspect. 105 (suppl. 3): 559-564, 1997.

8. Telang N T et al. Proc. Soc. Exp. Bio. Med. 216: 246-252, 1997

9. Telang N T et al. Brit. J. Cancer. 77:1549-1554, 1998

10. Katdare M et al. Ann NY Acad. Sci. 889:267-252, 1999

11. Jinno H et al. Carcinogenesis 20: 229-236, 1999

12. Kalabis J et al. Proc. Amer. Assoc. Cancer Res. 41: 173 (Abst # 1108), 2000.

13. Ohnishi T, Kousaku O Biotechniques 27: 978-985, 1999

14. Booth C et al. Epith. Cell Bio. 4: 78-86, 1995

15. Whitehead R H et al. In vitro Cell Dev. Biol. 23: 436-442, 1987

16. Whitehead R H Joseph J L Epith. Cell Biol. 3: 119-125, 1994

17. Sevignani C et al. J. Clin. Invest. 101: 1572-1580, 1998

18. Grossman J et al. Amer. J. Pathol. 153: 53-62, 1998

19. Sevignani C et al. Cancer Res. 59: 5882-5886, 1999

20. Fodde R et al. Proc. Natl. Acad. Sci. USA 91: 8969-8973, 1994

21. Oshima M et al. Proc. Natl. Acad. Sci. USA 92: 4482-4486, 1995.

22. Su L K et al. Science 256: 668-670, 1992

23. Moser A R et al. Science 247: 322-324, 1990

24. Bertagnolli M., Ann NY Acad. Sci. 847: 32-44, 1999

25. Boolbol S K et al. Cancer Res. 56: 2556-2560, 1996

26. Beazer-Barkley Y et al. Carcinogenesis 17: 1757-1760, 1996

27. Jacoby R F et al. Cancer Res. 60: 1864-1870, 2000

28. Saez E et al. Nature Med. 4: 1058-1061, 1998

29. Lefebvre A M et al. Nature Med. 4: 1053-1057, 1998

30. Zurcher C et al. The Mouse in Biomedical Research: Experimental Biology and Oncology. 4: 11-35, 1982, Academic Press, New York, N.Y.

31. Jacks T, Ann Rev. Genetics, 30: 603, 1996.

32. Groden J et al. Cell 66: 589-600, 1991.

33. Nishiho I et al. Science 253: 665-669, 1991

34. Lothe R A et al. Cancer Res 53: 5849-5852, 1993

35. Lynch H T et al. Gastroenterology 104; 1535-1549, 1993.

36. Edelmann W et al. Cancer Res. 59: 1301-1307, 1999

37. Zhu Y et al. Cell 94: 703-714, 1998

38. Song J et al. Cancer Res. 60: 3191-3199, 2000

39. Sasai S et al. Carcinogenisis 21:953-950, 2000

40. Paulsen J E et al. Cancer Res. 61:5010-5015, 2001

41. Moser A R et al. Cancer Res. 61:3480-3485, 2001

42. Sansom O J et al. Cancer Res. 61:7060-7064, 2001

What is claimed is:

1. A method for screening at least one test agent for chemopreventive efficacy, said method comprising the steps of:
(a) providing a subculturable epithelial cell line, wherein said subculturable epithelial cell line exhibits aberrant cellular proliferation;
(b) exposing cells of the subculturable epithelial cell line to the at least one test agent; and
(c) determining an effect of the at least one test agent;
wherein said subculturable epithelial cell line is derived from histologically normal non-cancerous epithelial tissue of a gene knock out mouse, said gene knock out mouse having an inactivated gene, wherein presence of said inactivated gene renders said gene knock out mouse susceptible to development of an epithelial cancer.

2. The method of claim 1 wherein the inactivated gene is a tumor suppressor gene.

3. The method of claim 2, wherein the tumor suppressor gene is adenomatous polyposis coli.

4. The method of claim 1 wherein the effect of the at least one test agent comprises modulating the number of cells exhibiting cellular responses indicative of a precancerous or cancerous state.

5. The method of claim 4, wherein said modulation is a decrease in the number of cells exhibiting a precancerous or cancerous state.

6. The method of claim 4 wherein the cellular responses indicative of said precancerous or cancerous state are selected from the group consisting of aneuploidy, telomerase re-expression, loss of contact inhibition and anchorage-independent growth.

7. The method of claim 1 wherein the subculturable epithelial cell line is derived from histologically normal noncancerous cells, said histologically normal noncancerous cells comprising at least one mutation in a tumor suppressor gene.

8. A method of claim 7 wherein the subculturable epithelial cell line displays at least one precancerous or cancerous marker selected from the group consisting of aneuploidy, telomerase re-expression, loss of contact inhibition and anchorage-independent growth.

9. The method of claim 1 wherein the cells of the subculturable epithelial cell line are preneoplastic.

10. The method of claim 8 wherein the subculturable epithelial cell line is an epithelial population of not more than 15 passages.

11. The method of claim 9 wherein the subculturable epithelial cell line is an epithelial population of not more than 15 passages.

12. A method of claim 8 wherein the subculturable epithelial cell line is an epithelial population of at least 15 passages.

13. A method of claim 9 wherein the subculturable epithelial cell line is an epithelial population of at least 15 passages.

14. A subculturable epithelial cell line, wherein said subculturable epithelial cell line is derived from histologically normal non-cancerous epithelial tissue of a gene knock out mouse, said gene knock out mouse having an inactivated gene, wherein presence of said inactivated gene renders said gene knock out mouse susceptible to development of an epithelial cancer and wherein the subculturable epithelial cell line is an epithelial population at least 5 passages.

15. The subculturable epithelial cell line of claim 14, wherein the inactivated gene is a tumor suppressor gene.

16. The subculturable epithelial cell line of claim 15, wherein the tumor suppressor gene is adenomatous polyposis coli.

17. The subculturable epithelial cell line of claim 14, wherein the subculturable epithelial cell line is an epithelial population of at least 15 passages.

18. The subculturable epithelial cell line of claim 14, wherein cells of the subculturable epithelial cell line have one or more mutations in a tumor suppressor gene.

19. A subculturable epithelial cell line of claim 14, wherein the subculturable epithelial cell line is derived from a knock out mouse having a genotype selected from the group consisting of Apc1638N[+/−] and Apc[+/+]C57COL.

20. A subculturable epithelial cell line of claim 14, wherein the subculturable epithelial cell line is an early passage cell line.

21. A subculturable epithelial cell line of claim 18, wherein the subculturable epithelial cell line is of gastrointestinal origin.

22. A subculturable epithelial cell line of claim 18, wherein the subculturable epithelial cell line is derived from colon.

23. A cell line which is a clonal derivative of a subculturable epithelial cell line of claim 18.

24. A cell line which is a clonal derivative of a subculturable epithelial cell line of claim 19.

25. A subculturable epithelial cell line of claim 24, wherein the cell line is Strang No. 1 Apc [+/−] 1638NCOL and derivatives thereof.

26. A subculturable epithelial cell line of claim 24, wherein the cell line is Strang No. 2 1638N-Cl₁, and derivatives thereof.

27. A subculturable epithelial cell of claim 24, wherein the cell line is Strang 1638N Pr₁ cells and derivatives thereof.

28. A subculturable epithelial cell line and derivatives thereof, wherein the cell line is Strang No. 4 Ape [+/+] C57COL cells, said cell line derived from normal colonic mucosal epithelium of a mouse, said mouse having an Apc[+/+] C57COL genotype.
